(19) United States
(12) Patent Application Publication
(10) Pub. No.: US 2018/0182488 A1
(43) Pub. Date: Jun. 28, 2018

(54) METHOD AND APPARATUS FOR PROVIDING A PREDICTION

(71) Applicant: Kirontech Holding SAL, Hazmieh, Baaba (LB)

(72) Inventor: Tarek NASSAR, Cambridge (GB)

(21) Appl. No.: 15/736,001

(22) PCT Filed: Jun. 15, 2015
(86) PCT No.: PCT/FI2015/050427

§ 371 Date: Dec. 13, 2017

(51) Int. Cl. G16H 50/20; G16H 10/60

(52) U.S. Cl. CPC G16H 50/20; G16H 10/60

(57) ABSTRACT

A computer implemented method includes analysing a dataset; receiving a request to provide a predicted field value for a certain target field of a certain target data record that, includes one or more explanatory fields; determining univariate counts indicative of value variation in the target field and explanatory fields across the dataset; determining bivariate counts indicative of value pair variation in field pairs comprising the target field and the explanatory fields across the dataset; using the univariate counts and bivariate counts for determining data record signatures for different target field values, wherein the signature includes explanatory field values; repeating the determining of signatures until certain predefined limit is reached; selecting a signature that at least partially matches values of explanatory fields of the target data record; and concluding that the predicted field value for the target field is the value of the target field corresponding to the selected signature.

### TECHNICAL FIELD

The present application generally relates to a computer implemented prediction method. The method is suited for example, though not limited to, the analysis of health data.

### BACKGROUND

This section illustrates useful background information without admission of any technique described herein as representative of the state of the art.

The amount and quality of big data collected is growing at a fast rate. Various technologies and innovations have focused on collection, storage and retrieval of big data but there is also a need for large scale, automated analysis of this data, in particular, the ability to use the collected data to make predictions.

### SUMMARY

Various aspects of the disclosed embodiments are set out in the claims.

According to a first example aspect of the present disclosure, there is provided a computer implemented method comprising:

analysing a dataset comprising a plurality of data records, wherein each data record comprises data fields, a data field comprising a field name and a field value;

receiving a request to provide a predicted field value for a certain target field of a certain target data record, wherein said certain target data record comprises one or more explanatory fields with explanatory values;

determining univariate counts indicative of value variation in said target field and in one or more explanatory fields across the dataset;

determining bivariate counts indicative of value pair variation in field pairs comprising said target field and at least one of the explanatory fields across the dataset;

using the univariate counts and bivariate counts for determining data record signatures for different values of the target field, wherein the signature comprises values of the explanatory fields;

repeating said determining of signatures until certain predefined limit is reached;

selecting a signature that at least partially matches values of explanatory fields of the target data record; and

concluding that the predicted field value for the target field is the value of the target field corresponding to the selected signature.

In an embodiment the method further comprises selecting a subset of the dataset to form a learning dataset comprising a plurality of records and determining the data record signatures for said learning dataset; and repeating the selecting a subset of the dataset and determining the data record signatures until a predefined limit is reached.

In an embodiment the method further comprises changing parameters used in determination of data record signatures and performing the determination of data record signatures with the changed parameters; and repeating the changing of the parameters and performing the determination of data record signatures until a predefined limit is reached.

In an embodiment the method further comprises pre-processing the dataset prior to analysing the dataset. The pre-processing comprises determining the univariate counts for each field in the dataset, determining the bivariate counts for certain data field pairs of records of the dataset, and storing the univariate counts and bivariate counts for future use.

In an embodiment determining the univariate counts comprises collecting distinct field values for each field in the dataset to obtain a value range for each field; and calculating for each field a total number of occurrences of each field value in the value range to obtain the univariate counts for each field.

In an embodiment determining the bivariate counts comprises processing the data fields of the data records in pairs by collecting distinct field value pairs for each field pair to obtain a bivariate range of the field pair; and calculating for each field pair a total number of occurrences of each field value in the bivariate value range to obtain bivariate counts for each field pair.

In an embodiment determining the data record signatures comprises using a function of the univariate and bivariate counts to determine scores indicative of likelihood of a certain value of the target field and certain values of the explanatory fields to exist in the same data record and using the scores to determine the signatures for different values of the target field.

In an embodiment determining the data record signatures comprises determining, for different target field values, a false predictions count indicative of the number of records in the dataset, which records comprise explanatory field names and explanatory field values of a certain signature but which records do not comprise the respective target field value, determining, for different target field values, a missed predictions count indicative of the number of records in the dataset, which records comprise a certain target field value but do not comprise the respective signature, and using a function of the false prediction count and missed prediction count for determining the data record signatures.

In an embodiment the request to provide the predicted field value comprises an indication of the explanatory fields to be used in said prediction.

In an embodiment the request to provide the predicted field value comprises said predefined limit for repeating said determining of signatures.

In an embodiment the predefined limit is certain number of iterations or certain score value for the signature.

According to a second example aspect of the present disclosure, there is provided a server apparatus comprising:

a processor; and

a memory including computer program code; the memory and the computer program code configured to, with the processor, cause the apparatus to

analyse a dataset comprising a plurality of data records, wherein each data record comprises data fields, a data field comprising a field name and a field value;

receive a request to provide a predicted field value for a certain target field of a certain target data record, wherein said certain target data record comprises one or more explanatory fields with explanatory values;

determine univariate counts indicative of value variation in said target field and in one or more explanatory fields across the dataset;

determine bivariate counts indicative of value pair variation in field pairs comprising said target field and at least one of the explanatory fields across the dataset;

use the univariate counts and bivariate counts for determining data record signatures for different values of the target field, wherein the signature comprises values of the explanatory fields;

repeat said determining of signatures until certain predefined limit is reached;

select a signature that at least partially matches values of explanatory fields of the target data record; and

conclude that the predicted field value for the target field is the value of the target field corresponding to the selected signature.

In an embodiment the memory and the computer program code are further configured to, with the processor, cause the apparatus to

select a subset of the dataset to form a learning dataset comprising a plurality of records and determining the data record signatures for said learning dataset;

repeat the selecting a subset of the dataset and determining the data record signatures until a predefined limit is reached.

In an embodiment the memory and the computer program code are further configured to, with the processor, cause the apparatus to

change parameters used in determination of data record signatures and performing the determination of data record signatures with the changed parameters;

repeat the changing of the parameters and performing the determination of data record signatures until a predefined limit is reached.

In an embodiment the memory and the computer program code are further configured to, with the processor, cause the apparatus to pre-process the dataset prior to analysing the dataset, wherein the pre-processing comprises

determining the univariate counts for each field in the dataset,

determining the bivariate counts for certain data field pairs of records of the dataset, and

storing the univariate counts and bivariate counts for future use.

In an embodiment the memory and the computer program code are further configured to, with the processor, cause the apparatus to determine the univariate counts by

collecting distinct field values for each field in the dataset to obtain a value range for each field; and

calculating for each field a total number of occurrences of each field value in the value range to obtain the univariate counts for each field.

In an embodiment the memory and the computer program code are further configured to, with the processor, cause the apparatus to determine the bivariate counts comprises processing the data fields of the data records in pairs by

collecting distinct field value pairs for each field pair to obtain a bivariate range of the field pair; and

calculating for each field pair a total number of occurrences of each field value in the bivariate value range to obtain bivariate counts for each field pair.

In an embodiment the memory and the computer program code are further configured to, with the processor, cause the apparatus to determine the data record signatures by

using a function of the univariate and bivariate counts to determine scores indicative of likelihood of a certain value of the target field and certain values of the explanatory fields to exist in the same data record and using the scores to determine the signatures for different values of the target field.

In an embodiment the memory and the computer program code are further configured to, with the processor, cause the apparatus to determine the data record signatures by

determining, for different target field values, a false predictions count indicative of the number of records in the dataset, which records comprise explanatory field names and explanatory field values of a certain signature but which records do not comprise the respective target field value,

determining, for different target field values, a missed predictions count indicative of the number of records in the dataset, which records comprise a certain target field value but do not comprise the respective signature, and

using a function of the false prediction count and missed prediction count for determining the data record signatures.

According to a third example aspect of the present disclosure, there is provided a computer program comprising computer executable program code configured to control an apparatus, when the computer executable program code is executed, to:

analyse a dataset comprising a plurality of data records, wherein each data record comprises data fields, a data field comprising a field name and a field value;

receive a request to provide a predicted field value for a certain target field of a certain target data record, wherein said certain target data record comprises one or more explanatory fields with explanatory values;

determine univariate counts indicative of value variation in said target field and in one or more explanatory fields across the dataset;

determine bivariate counts indicative of value pair variation in field pairs comprising said target field and at least one of the explanatory fields across the dataset;

use the univariate counts and bivariate counts for determining data record signatures for different values of the target field, wherein the signature comprises values of the explanatory fields;

repeat said determining of signatures until certain predefined limit is reached;

select a signature that at least partially matches values of explanatory fields of the target data record; and

conclude that the predicted field value for the target field is the value of the target field corresponding to the selected signature.

In an embodiment the computer program comprises computer executable program code configured to control an apparatus, when the computer executable program code is executed, to perform any one of embodiments disclosed in relation to the first aspect.

Different non-binding example aspects and embodiments of the present disclosure have been illustrated in the foregoing. The embodiments in the foregoing are used merely to explain selected aspects or steps that may be utilized in implementations of the present disclosure. Some embodiments may be presented only with reference to certain example aspects of the present disclosure. It should be appreciated that corresponding embodiments may apply to other example aspects as well.

### BRIEF DESCRIPTION OF THE DRAWINGS

For a more complete understanding of example embodiments of the present disclosure, reference is now made to the following descriptions taken in connection with the accompanying drawings in which:

FIG. 1A shows a block diagram of an apparatus of an example embodiment;

FIG. 1B shows a flow diagram of a method of an example embodiment;

FIG. 2 shows an overall schema of a method of an example embodiment;

FIG. 3 shows a method of data structuring according to an example embodiment;

FIG. 4 shows a method of data loading according to an example embodiment;

FIG. 5 shows a method of formulating a question according to an example embodiment;

FIG. 6 shows a method of bucketing according to an example embodiment;

FIG. 7 shows a method of pre-processing data according to an example embodiment; and

FIG. 8 shows a method of signature selection according to an example embodiment.

### DETAILED DESCRIPTION OF THE DRAWINGS

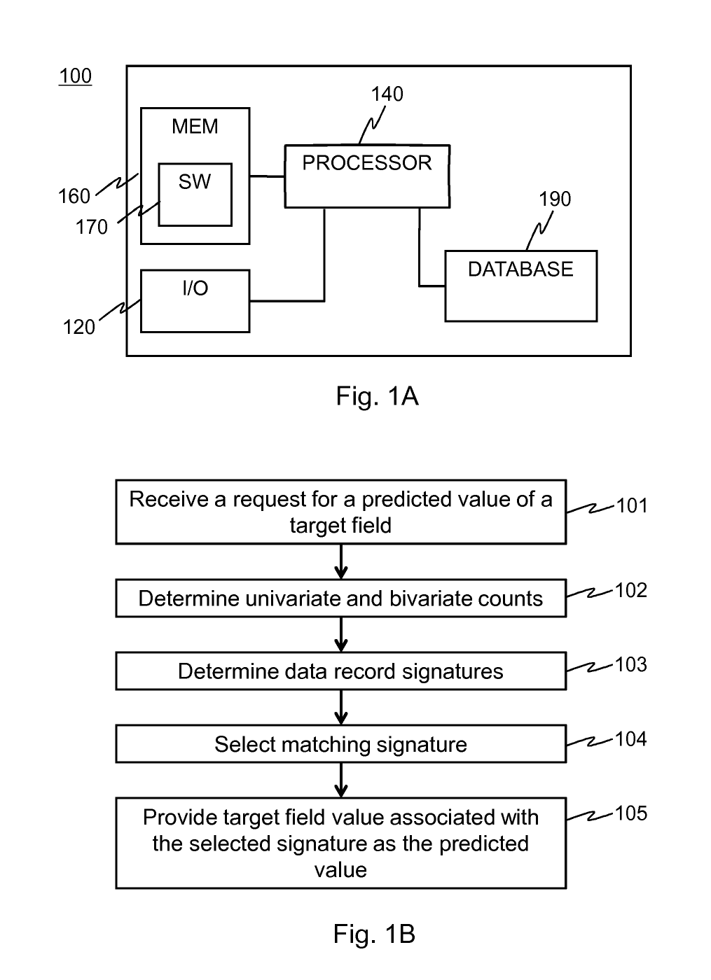

Example embodiments of the present disclosure and potential advantages are understood by referring to FIGS. 1A through 8 of the drawings. In this document, like reference signs denote like parts or steps.

Various embodiments of the present disclosure provide methods for analysing large amounts of data (referred to as big data) and making predictions based on the large amounts of data. A large collection of data refers to such amount of data that traditional data processing applications do not necessarily suit for analysing the data (in a reasonable time frame).

In an embodiment there is provided a computer-implemented prediction system based on machine learning geared to automated analysis of data and for providing predictions based on the data.

In the following examples various embodiments of the present disclosure are discussed in connection with healthcare data and healthcare applications. However, the embodiments of the present disclosure may be applied to other types of data too. In general embodiments of the present disclosure are suited for analysis of big data (large data sets) of any kind and for making predictions based on the analysis.

For example in healthcare and pharmaceutical/biotech sectors there is a problem of how to extract predictions and practically useful knowledge from heaps of available data. Analysis of available data may require a lot of work from data scientists, IT professionals and medical or pharmaceutical experts. In order to make better use of available data, there is a need to allow health care practitioners, researchers and pharmaceutical scientists to easily extract knowledge from data without the need to write code or understand complex statistical and mathematical techniques. There is also a need to make predictions based on the available data.

In an embodiment there is provided a method and a system that searches through longitudinal population-wide healthcare databases for patterns that apply to the patient. In an embodiment, a novel and inventive method is used for finding the patterns that apply to the patient. In an embodiment, faced with a particular combination of symptoms, lab test results, health record history and/or genomic data for a given patient, the method of an embodiment of the present disclosure may predict or recommend a certain diagnosis or preferred treatment. Such a prediction or a recommendation may then be submitted to the treating physician for his/her expert opinion.

After a recommendation has been made, the system may keep track of whether it has been accepted or rejected by the physician and on the eventual medical outcome. This feedback may be used for continuous learning and improvement of the system.

Patterns in biological data can vary from very simple assertions such as “high insulin levels are indicative of diabetes” to more complex assertions such as “high estrogen, Ki67 and old age significantly increase the mortality rates in breast cancer patients”. Given the inter-connectedness, complexity and number of variables involved in biological data, the sheer number of such patterns is too large to be reasonably analyzed through a brutal force approach. In an embodiment there is provided a method (a machine learning algorithm) that proceeds by eliminating a priori patterns with low probability and then checks the data only for high probability patterns. In this way a smart navigation is provided through the set of all possible patterns to reach the most probable patterns.

FIG. 1A shows a block diagram of an apparatus 100 in which various embodiments of the invention may be applied. The apparatus is for example a general-purpose computer or server or some other electronic data processing apparatus.

The general structure of the server apparatus 100 comprises a processor 140, and a memory 160 coupled to the processor 140. The apparatus 100 further comprises software 170 stored in the memory 160 and operable to be loaded into and executed in the processor 140. The software 170 may comprise one or more software modules and can be in the form of a computer program product. Further, the apparatus 100 comprises an input/output unit 120 and a database unit 190 coupled to the processor 140.

The processor 140 may be, e.g., a central processing unit (CPU), a microprocessor, a digital signal processor (DSP), a graphics processing unit, or the like. FIG. 1 shows one processor 140, but the apparatus 100 may comprise a plurality of processors.

The memory 160 may be for example a non-volatile or a volatile memory, such as a read-only memory (ROM), a programmable read-only memory (PROM), erasable programmable read-only memory (EPROM), a random-access memory (RAM), a flash memory, a data disk, an optical storage, a magnetic storage, a smart card, or the like. The apparatus 100 may comprise a plurality of memories. The memory 160 may be constructed as a part of the apparatus 100 or it may be inserted into a slot, port, or the like of the apparatus 100 by a user. The memory 160 may serve the sole purpose of storing data, or it may be constructed as a part of an apparatus serving other purposes, such as processing data.

The input/output unit 120 may comprise communication modules that implement data transmission to and from the apparatus. The communication modules may comprise, e.g., a wireless or a wired interface module. The wireless interface may comprise such as a WLAN, Bluetooth, infrared (IR), radio frequency identification (RF ID), GSM/GPRS, CDMA, WCDMA, or LTE (Long Term Evolution) radio module. The wired interface may comprise such as Ethernet or universal serial bus (USB), for example. Further the input/output unit 120 may comprise a user interface for providing interaction with a user of the apparatus. The user interface may comprise a display and a keyboard, for example. The user interaction may be implemented through communication modules too.

The database unit 190 is configured for storing data that is used for analysis and predictions in embodiments of the present disclosure. The database unit 190 may be a separate component or certain memory area in the memory 160 or the database unit 190 may be located in a physically separate database server that is accessed for example through the communication interface of the input/output unit 120. The database unit 190 may be a relational (e.g. SQL) or a non-relational (e.g. NoSQL) database.

A skilled person appreciates that in addition to the elements shown in FIG. 1A, the apparatus 100 may comprise other elements, such as microphones, displays, as well as additional circuitry such as memory chips, application-specific integrated circuits (ASIC), other processing circuitry for specific purposes and the like. Further, it is noted that only one apparatus is shown in FIG. 1A, but the embodiments of the present disclosure may equally be implemented in a cluster of shown apparatuses.

In the following, certain terms used in this document are defined.
1. Record: A record is a data structure labeled uniquely by a code known as the unique identifier and which contains one or multiple fields.
2. Dataset: A collection of records. The different records in a given dataset need not have the same structure, i.e. they do not necessarily have the same fields.
3. Learning dataset: A subset of records of a full dataset. The learning dataset is used for making a prediction.
4. Out of sample dataset: A subset of records of the full dataset. An out of sample dataset comprises records that are not contained in the learning dataset.
5. Field: A field is a data structure which is a couple consisting of a field name and a field value. Within a given record, a field name can occur only once. However, the same field name may be associated with multiple values.
6. Field type: A field type refers to the domain of the field values. The possible domains are:
   a. Numerical:
      i. Discrete, i.e. where the field values are taken from the set of natural numbers.
      ii. Continuous, i.e. where the field values are taken from the set of real numbers.
   b. Categorical, i.e. where the field values are taken from a finite alphabet of symbols.
7. Field category: A field value can be a single variable, a set of variables or a list of variables. A set of variables is a collection of more than one variable where the order in which these variables appear is not important, i.e. {a,b} is the same as {b,a}. A list of variables is a collection of more than one variable where the order of the variables is important, i.e. [a,b] is different from [b,a].
8. Predicted field or a target field: a field name whose value we wish to predict based on some prior information.
9. Predicted value: the predicted value of the predicted field.
10. Explanatory variables: the collection of all field names whose values may be used as prior information.
11. Explanatory field: a couple consisting of a field name and a field value which may be used in prediction.
12. Signature: A subset of the explanatory fields that is ultimately chosen by the system to reach a specific predicted value for a predicted field.

In the following, some examples that are useful for understanding the embodiments of the present disclosure are given.

RECORD
a. Tabular record:

| Unique Identifier | Gender | Age | Progesterone level | Estrogen level |
|---|---|---|---|---|
| Hht166718 | Male | 37 | 92 | 98 |

b. Json record:

```
{
  “unique_id”: “Hht166718”,
  “Gender”: “Male”,
  “Age”: 37,
  “Ki67”: 1,
  “Estrogen”: 98
}
```

DATASET
a. Tabular data where each record has the same structure

| Unique Identifier | Gender | Age | Progesterone level | Estrogen level |
|---|---|---|---|---|
| Hht166718 | Male | 37 | 92 | 98 |
| 1iuqiw89991 | Female | 18 | 12 | 85 |
| #4415271H | Female | 79 | 66 | 14 |

b. Json data where records have different structure

```
[
  {
    “unique_id”: “Hht166718”,
    “Gender”: “Male”,
    “Age”: 37,
    “Ki67”: 1,
    “Estrogen”: 98
  },
  {
    “unique_id”: “liuqiw89991”,
    “Gender”: “Female”,
    “Age”: 18,
    “Estrogen”: 85
  },
  {
    “unique_id”: “#4415271H”,
    “Gender”: “Female”,
    “Age”: 79,
    “Progesterone”: 66,
    “Estrogen”: 14
  }
]
```

FIELD
a. Tabular field
Gender
Male
b. Json field
{“Estrogen”: 14}
FIELD TYPE

a. Numerical discrete: {“Number of lymph nodes”: 9 }
b. Numerical continuous: {“Estrogen level”: 73.5561772}
c. Categorical: {“Species”: “Homo sapiens”}

FIELD CATEGORY

a. Singletons: “Organ”: “liver”
b. Sets: “Molecular function”: {“G01192881”, “G08872172”}
c. Lists: “DNA Sequence”: [GGTCAAAGTTWUQAA...]

PREDICTED FIELD, PREDICTED VALUE, EXPLANATORY VARIABLES, SIGNATURE

- Predicted/target field and value: {“mortality rate”: 0.23}
- Explanatory variables: {“estrogen level”, “progesterone level”, “age”,
“gender”, “Ki67”, “Her I”, “number of lymph nodes”}
- Signature: {“estrogen level”: 92, “Ki67”: 11}

It is to be noted that in an embodiment, the signature is associated with a specific value for the predicted field, i.e. a mortality rate of 0.23 is best explained by an estrogen level of 92 combined with a Ki67 of 11. Different mortality rates may be best explained by completely different signatures that may not even contain the same field names as the signature which explains a mortality rate of 0.23.

Further it is to be noted that in an embodiment, the signature is not unique, i.e. we may have different signatures, which explain the same predicted field value.

FIG. 1B shows a flow chart of a method of an example embodiment. The method may be performed e.g. in the apparatus of FIG. 1A. A dataset comprising a plurality of data records is analysed. Each data record of the dataset comprises data fields, and a data field comprises a field name and a field value. The data set may be a learning dataset randomly or otherwise selected from a larger set of data. The method comprises the following phases:

101: Receive a request for a predicted field value for a certain target field of a certain target data record. The target data record comprises one or more explanatory fields with explanatory values. The request may define which explanatory fields should be used for making the prediction.

102: Determine univariate and bivariate counts across the dataset. In an embodiment the univariate and bivariate counts are determined and stored beforehand, prior to receiving a request for predicted value. Univariate counts are indicative of value variation in said target field and in one or more explanatory fields across the dataset and bivariate counts are indicative of value pair variation in field pairs comprising said target field and at least one of the explanatory fields (or simply other fields) across the dataset.

103: Determine data record signatures for different values of the target field. The univariate counts and bivariate counts are used for this. The signature comprises values of the explanatory fields and an associated value of the target field. It is noted that data record signatures of different values of the same target field may comprise values for different explanatory fields. Determining of the signatures may be repeated until certain predefined limit is reached. The limit may be for example certain number of iterations or certain score value of the signature and the limit may be given in the request for the predicted value.

104: Select a signature that matches the target data record. For example, select the signature so that at least some of the explanatory field values of the signature match values of the explanatory fields of the target data record. The precision that is to be used may be defined in the request for the predicted value.

105: Provide target field value associated with the selected signature as the predicted value.

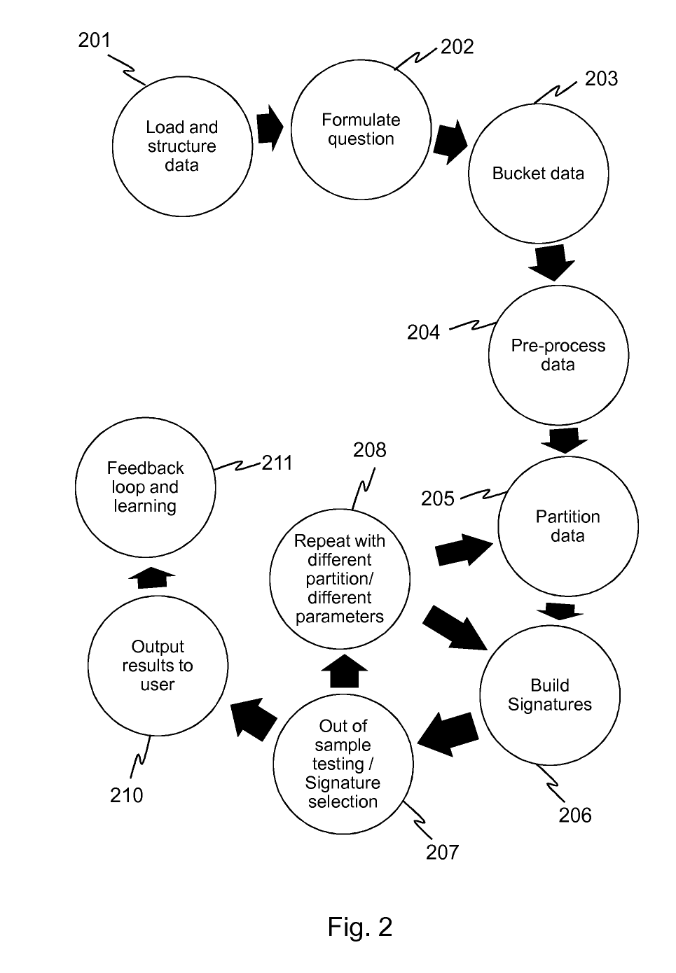

FIG. 2 shows an overall schema of a method of an example embodiment. The method comprises the following phases:

201: Load and structure data. The dataset, which may be available in raw form, e.g. text files, comma separated files etc. . . . , is transformed/structured into a form where it can be loaded into a relational (SQL) or non-relational (NoSQL) database. The structured data is uploaded to a computer or many computers forming a cluster.

202: The question is formulated. The user of the system selects a specific variable to be predicted, e.g. “mortality rate”. The user may specify which other fields are to be used as the explanatory fields in the analysis. If the user does not specify which other fields should be used as the explanatory fields, then by default all fields present in the dataset may be considered eligible explanatory fields. The user may also specify the accuracy to which he/she wants the prediction to proceed.

203: The data is bucketed if needed.

204: The dataset may be pre-processed. In an embodiment the pre-processing phase comprises processing of field values in the data records and for example forming a histogram of different values in the fields. In an embodiment the pre-processing phase comprises determining univariate and bivariate counts and storing the determined values for future use. In an embodiment the pre-processing comprises:
1. For each field name in the dataset, collect all the distinct field values it can take. If the field type is a set, then treat each element of that set as a field value and include it in the collection process. The collection of all distinct field values for a given field name will be referred to as the “range of the field”.
2. Calculate univariate counts, i.e. for each field name, calculate the total number of occurrences of each field value in the range of the field across the records in the dataset.
3. For each pair of field names, collect all the distinct field value pairs corresponding to said pair of field names. If one or both of the field types is a set, then the Cartesian product of the field values and treat each element of the Cartesian product as a value pair in the collection process. The resulting set of distinct field value pairs for the each chosen pair of field names will be referred to as the “bivariate range of the field names pair”.
4. Calculate bivariate counts, i.e. for each pair of field names, calculate the total number of occurrences of every element in the bivariate range of the field names pair across the records in the dataset.

205: The dataset is partitioned. In an example a certain subset of the full dataset is selected for processing. For example a random subset may be selected for being used as a learning dataset for providing the prediction.

For example, if the full dataset comprises one million records, a learning dataset of ten thousand or hundred thousand records may be chosen. These are however only example numbers and the size of the full dataset and the size of the learning dataset may be something else too.

206: Signatures are built. The univariate and bivariate counts are used for this.

In an embodiment the signatures are built using a function of the univariate and bivariate counts to determine scores indicative of likelihood of a certain value of the target field and certain values of the explanatory fields to exist in the same data record. In an embodiment the function is recursively minimized to obtain the scores. The scores are used for determining the signatures for different values of the target field.

In an embodiment the signatures are built as follows:

We will use the following notation. Let P be the predicted/target field name and p be some field value for P. Let E be some field name distinct from P and let e be some field value for E. Now denote by:

N(P=p): the univariate count of the field value p corresponding to the field name P.
N(E=e): the univariate count of the field value e corresponding to the field name E.
N(P=p, E=e): the bivariate count of the pair of field names (P,E) with field values (p,e).
1. For the selected target field P and value p, minimize the following score:

### A) Score(P=p, E=e)=N(E=e)−N(P=p, E=e)+L*(N(P=p)−N(P=p, E=e))

Where the minimum is taken over all possible field names E and corresponding field values e and the parameter L is a number between 0 and 1 called the “penalty parameter”.
2. Denote by E*=e* the field which minimizes the score in A).
   Now compute the quantities:
   N(P=p, E*=e*): the bivariate count of the fields P=p and E*=e*.
   N(E=e): the univariate count of the field value e corresponding to the field name E where
   E is not the same as E* or, if E is the same as E* then e is different from e*.
   N(P=p, E*=e*, E=e): the trivariate count of the triplet of field names (P,E*,E) with field values (p,e*,e).
3. Minimize over all values e of E and over all possible field names E the following score:

### B) Score(P=p, E*=e*, E=e)=N(E=e)=N(P=p, E*=e*, E=e)+L*(N(P=p)−N(P=p, E*=e*,E=e))

Let E** and e** be the field name and field value for which the score B) is minimal.
4. Repeat step 2 where every occurrence of E*=e*, where E=e is replaced by E**=e**, E*=e*, E=e and so on recursively until either:
   a. A certain pre-specified number of iterations is reached or,
   b. A certain pre-specified value for the score is reached
   c. In case b, i.e. if the pre-specified value for the score is never reached, then return the last signature obtained until it is no longer possible to proceed further with the recursion.

In another embodiment, the signatures are built as follows. First of all, a false predictions count and a missed predictions count are defined.
False predictions count: The number of records in the dataset which have the field names and values comprised in certain signature but which do not comprise the predicted/target field value for the predicted/target field. Example: Suppose that the signature {“Gender”: “Male”, “University”: “Aalto”} says predict “Engineer” for the field “Profession” if the record contains this signature. For every record in the data set where this prediction is incorrect, the “false predictions count” is increased by one.
Missed predictions count: The number of records which comprise the value of the predicted/target field but do not contain the respective signature. These are “missed” predictions because the predictor will not find them. It is understood that these are different from “False predictions count”.

A function of the false prediction count and missed prediction count is then used for determining the data record signatures. In an embodiment the function is recursively minimized.

In an embodiment the function of the false prediction count and missed prediction count is any numerical function “S” which exhibits the following properties: 1. It is strictly increasing in the argument “False predictions count” 2. It is strictly increasing in the argument “Missed predictions count” 3. It is bounded from above, i.e. it cannot go to infinity for any finite values of the arguments “False predictions count” and “Missed predictions count” 4. It is never zero unless both arguments “False predictions count” and “Missed predictions count” are zero. The signatures can be generated by recursively minimizing this function S (or equivalently, by maximizing any decreasing function of S) for example as described in the foregoing recursive minimization process.

The final set of explanatory fields in the foregoing example, i.e. E*=e*, E**=E***=e*** etc., is the signature we are looking for in the following sense: given a new record which was not in the analyzed dataset and for which the value of the target field P is not known, if that record contains this particular signature, then we will predict that the value of the predicted field P for this record will be p.

207: Signatures that have been obtained on the basis of the data partition that is being processed are reviewed. In an embodiment it is checked if the signatures that have been built for the learning dataset fulfil certain predefined criteria.

An out of sample dataset may be used for this purpose. The out of sample dataset refers herein to a set of data records that are not part of the learning dataset. The signatures are tested against the data of the out of sample dataset to see, if the signatures apply to the out of sample dataset too.

If the predefined criteria is not fulfilled, the process proceeds to repeating signature generation in phase 208.

If the predefined criteria is fulfilled, the process proceeds to providing results based on the signatures in phase 210.

208: If it is concluded that the signature generation will be repeated, the signature generation may be repeated for a different learning dataset and/or the parameters used in the signature generation may be changed. The process returns to phase 205 to select another partition of the dataset and/or the signatures are build with different parameters in phase 206. Phases 205-207 or 206-207 are may be iterated as many times as needed.

In an example, if the previous learning dataset comprised ten thousand records, a new learning dataset of twenty thousand records may be chosen. In an example, the new learning dataset may comprise partially or fully the same records as the previous learning dataset. In an example, the new learning dataset may comprise different records than the previous learning dataset. In an example, the L parameter in the score function may be changed.

210: Once it is concluded that suitable or sufficiently good signatures have been found, the prediction results are formed and output to the user of the system.

211: Optionally there may be a feedback loop. The user may for example give feedback on accuracy of the prediction.

It is noted that the order of different phases in FIG. 2 may vary. For example, the bucketing and pre-processing may be performed prior to formulating the question. Similarly, some of the shown phases may be left out from a particular implementation.

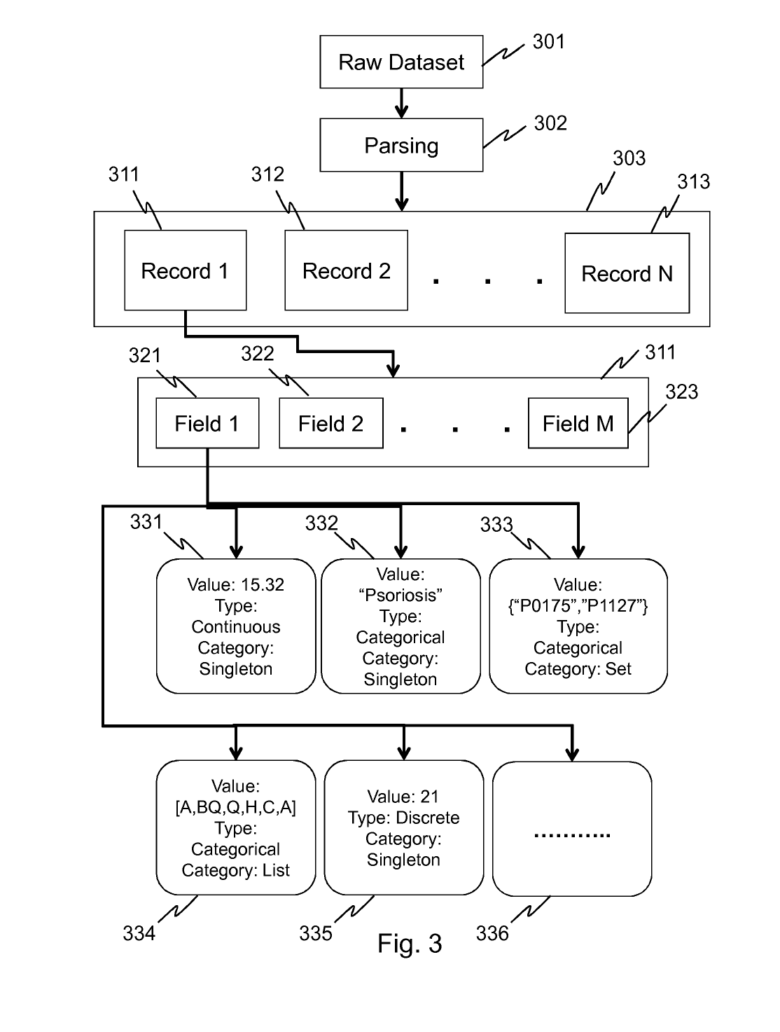

FIG. 3 shows a method of data structuring according to an example embodiment.

301: A raw dataset (e.g. e.g. text files, comma separated files etc. . . . ) is received in a processing functionality.

302: The data is parsed to suitable format for further processing.

Parsed dataset 303 comprises data records 1-N 311-313. Each data record comprises data fields. FIG. 3 shows data fields 1-M 321-323 of the data record 311. Each data field comprises a field name and a field value. FIG. 3 shows different types of data fields 331-336. Characteristics of example data field 331 comprise value: 15.23, type: continuous, and category: singleton. Characteristics of example data field 332 comprise value: “Psoriasis”, type: categorical, and category: singleton. Characteristics of example data field 333 comprise value: (“P0175”,“P1127”), type: categorical, and category: set. Characteristics of example data field 334 comprise value: [A,BQ,Q,H,C,A], type: categorical, and category: list. Characteristics of example data field 335 comprise value: 21, type: discrete, and category: singleton. It is noted that only some examples are given. Also other data field types are possible.

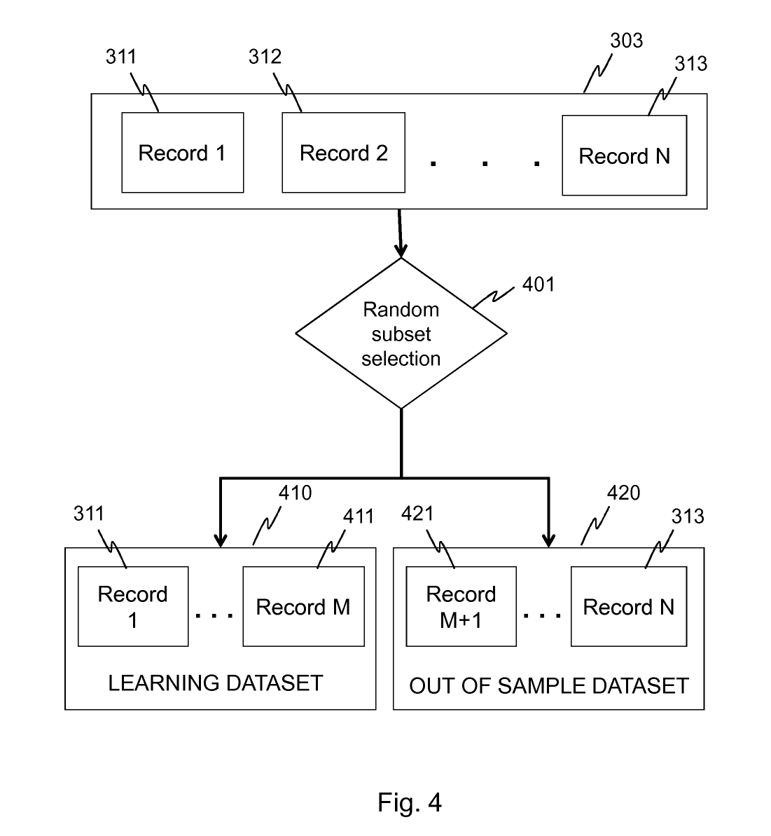

FIG. 4 shows a method of data loading according to an example embodiment. A structured dataset 303 that comprises data records 1-N 311-312 is processed. A subset of the dataset 303 is selected in phase 401. The selection may be random. As a result a learning dataset 410 is selected. In an example, the learning dataset comprises data records 1-M 311-411. Remaining dataset forms an out of sample dataset 420 that comprises data records M+1-N 421-313. In an embodiment, the out of sample dataset serves to test how good the signatures are when the signatures are applied to something new, i.e. a dataset different from the one used to reach them in the first place.

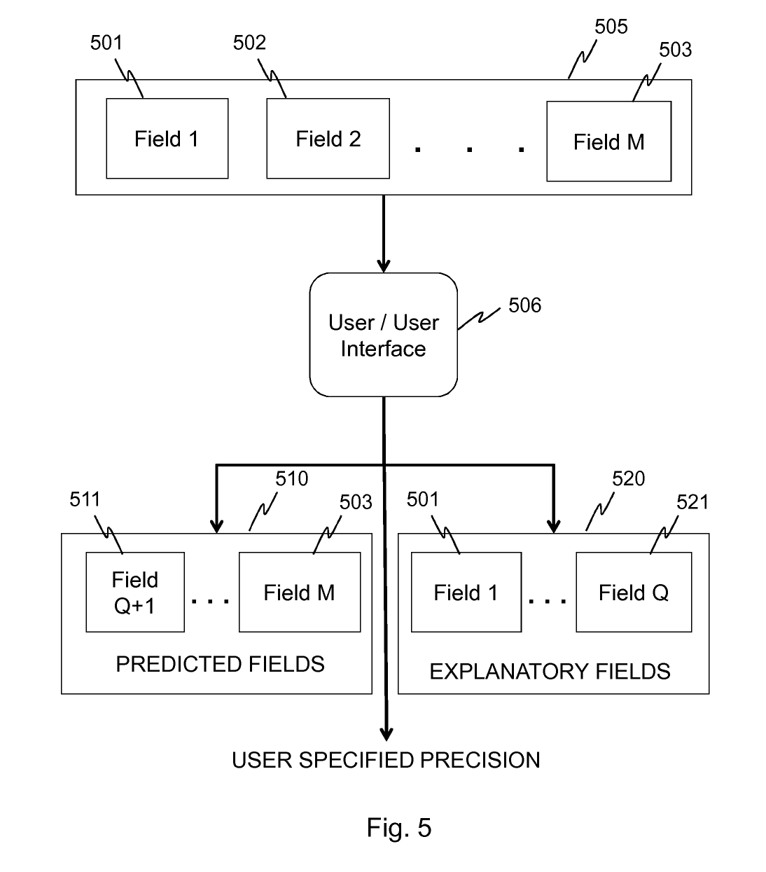

FIG. 5 shows a method of formulating a question according to an example embodiment.

Initially there is a target data record 505 that comprises data fields 1-M 501-503. Values of some of the data fields 501-503 are known and values of some of the data fields 501-503 are to be predicted. A user of the system defined which values (one or more values) are to be predicted though a user interface 506. In an example, data fields Q+1-M 511-503 are defined as predicted fields for which values are to be predicted. Data fields 1-Q 501-521 are defined as explanatory fields 520 that are to be used for providing the values for the predicted fields. The user of the system may also specify precision that is to be used for providing the predicted values.

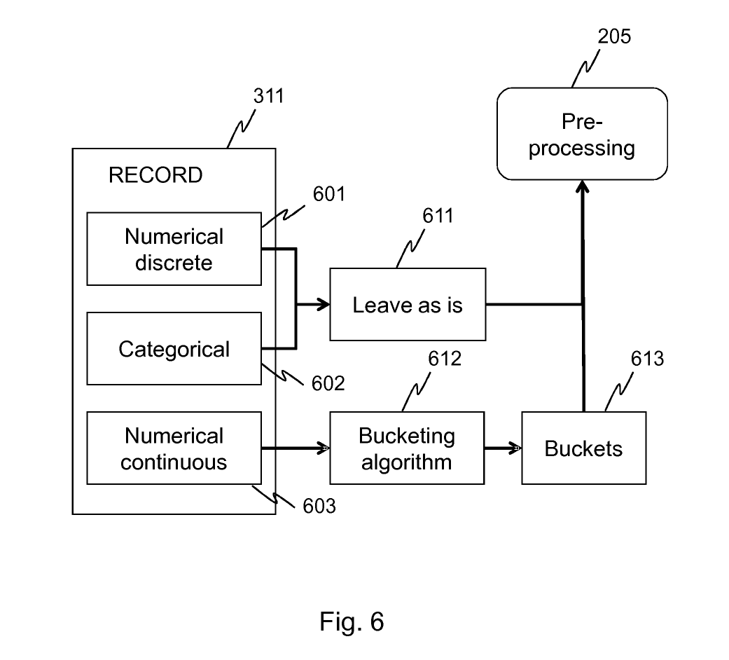

FIG. 6 shows a method of bucketing according to an example embodiment.

In an embodiment content of some of data fields of a record may be bucketed before further processing. FIG. 6 shows an example of providing a data record 311 to a pre-processing phase 205. The data record 311 comprises a numerical discrete data field 601, a categorical data field 602 and a numerical continuous data field 603. Data fields 601 and 602 are left as they are in phase 611 and thereby provided to the pre-processing phase as they are. The numerical continuous field 603 is processed into buckets 613 (numerical values with minor difference are put into the same basket) by a bucketing algorithm 612 and the bucketed data is provided to the pre-processing phase 205. Bucketing is useful when numerical values such as 0.111 and 0.1111, which although mathematically distinct, may be considered identical for the purpose of the prediction problem at hand. This can due to a multitude of reasons, for example (but not limited to):
1. The numerical difference 0.1111−0.111=0.0001 may simply be due to measurement error
2. The impact of this numerical difference is too small to change the outcome of the prediction etc. . . .

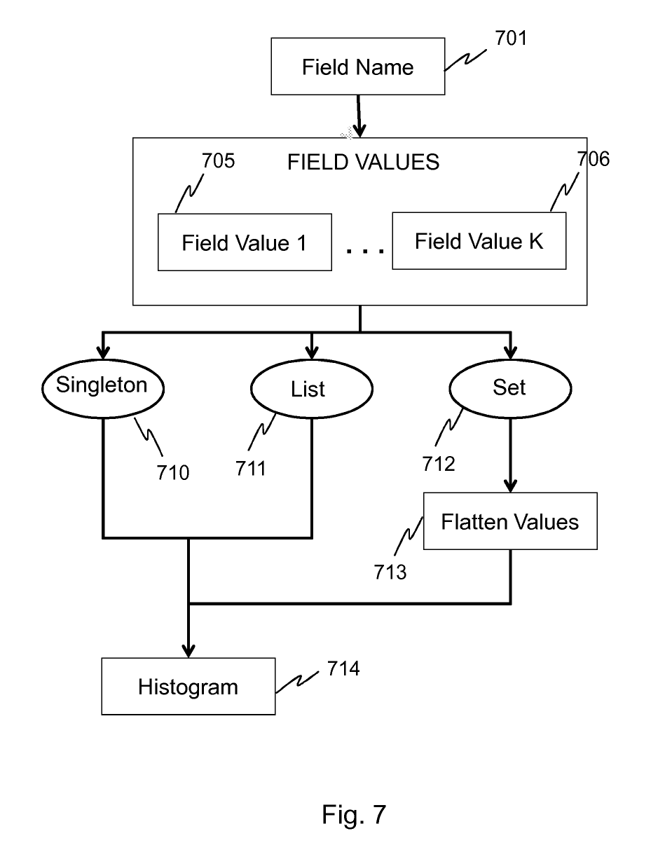

FIG. 7 shows a method of pre-processing data according to an example embodiment.

A certain field 701 with a certain field name is processed. The field comprises a plurality of field values 705-706. A histogram of different field values is formed 714. If category of the field value is singleton 710 or list 711 the field values are taken directly into the histogram. If category of the field value is set 712, the values of the set are first flattened 713 and then taken into the histogram.

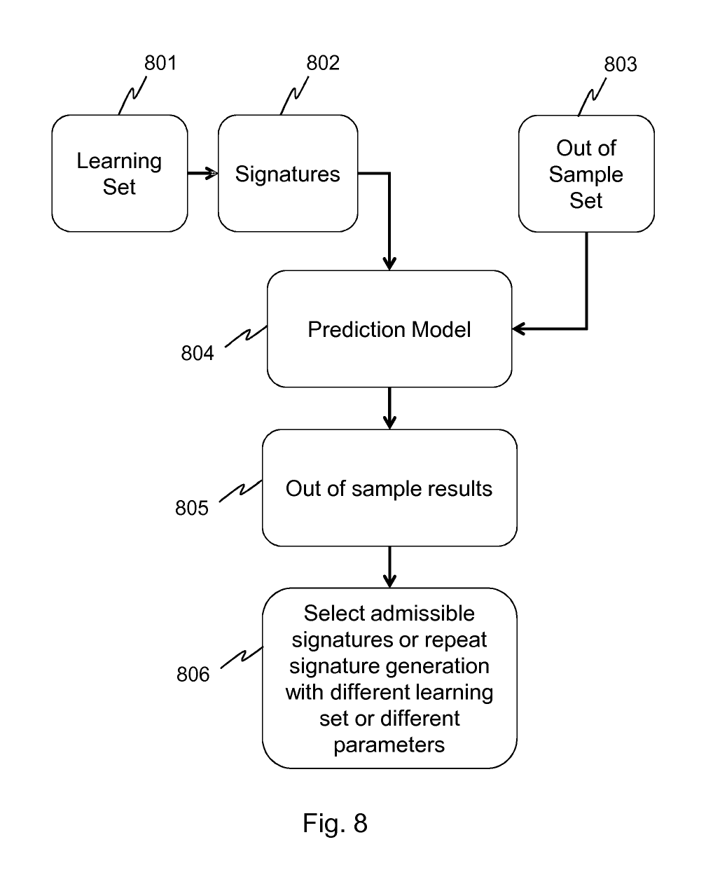

FIG. 8 shows a method of signature selection according to an example embodiment.

801: A learning set is processed. The learning set may be for example a random subset of an original dataset.

802: Signatures for the learning set are determined.

803: An out of sample set is supplied to the prediction model formed by the signatures determined for the learning dataset.

804: The signatures of the prediction model are tested using the data in the out of sample dataset.

805: The results of the out of sample testing are reviewed and on the basis of this it is determined whether the signatures of the prediction model fulfil certain predefined criteria, e.g. predefined precision set by the user of the system.

806: Admissible signatures are selected. For example signatures that fulfil predefined criteria are selected. In an embodiment signatures with certain score are selected. Alternatively the signature generation may be repeated with different learning set or with different parameters. For example larger learning set may be used or full different partition of the original dataset may be used.

Without in any way limiting the scope, interpretation, or application of the claims appearing below, a technical effect of one or more of the example embodiments disclosed herein is improved method and apparatus for analysing large amounts of data and/or for making predictions based on large amounts of data. For example, it may be possible to perform the analysis faster than previously as large amounts of data can be processed efficiently. Such data analysis cannot be implemented by simply using pen and paper due to large amount of data.

Another technical effect of one or more of the example embodiments disclosed herein is improved analysis of healthcare data that may help physicians diagnose and treat patients faster and better. Another technical effect of one or more of the example embodiments disclosed herein is improved cost structure.

If desired, the different functions discussed herein may be performed in a different order and/or concurrently with each other. Furthermore, if desired, one or more of the before-described functions may be optional or may be combined.

Although various aspects of the present disclosure are set out in the independent claims, other aspects of the present disclosure comprise other combinations of features from the described embodiments and/or the dependent claims with the features of the independent claims, and not solely the combinations explicitly set out in the claims.

It is also noted herein that while the foregoing describes example embodiments of the present disclosure, these descriptions should not be viewed in a limiting sense. Rather, there are several variations and modifications, which may be made without departing from the scope of the present disclosure as defined in the appended claims.

1.-21. (canceled)

22. A computer implemented method comprising:
analysing a dataset comprising a plurality of data records, wherein each data record comprises data fields, a data field comprising a field name and a field value;
receiving a request to provide a predicted field value for a certain target field of a certain target data record, wherein said certain target data record comprises one or more explanatory fields with explanatory values;
determining univariate counts indicative of value variation in said target field and in one or more explanatory fields across the dataset;
determining bivariate counts indicative of value pair variation in field pairs comprising said target field and at least one of the explanatory fields across the dataset;
using the univariate counts and bivariate counts for determining data record signatures for different values of the target field, wherein the signature comprises values of the explanatory fields and wherein determining the data record signatures comprises
determining, for different target field values, a false predictions count indicative of the number of records in the dataset, which records comprise explanatory field names and explanatory field values of a certain signature but which records do not comprise the respective target field value,
determining, for different target field values, a missed predictions count indicative of the number of records in the dataset, which records comprise a certain target field value but do not comprise the respective signature, and
using a function of the false prediction count and missed prediction count for determining the data record signatures;
repeating said determining of signatures until certain predefined limit is reached;
selecting a signature that at least partially matches values of explanatory fields of the target data record; and
concluding that the predicted field value for the target field is the value of the target field corresponding to the selected signature.

23. The method of claim 22, further comprising
selecting a subset of the dataset to form a learning dataset comprising a plurality of records and determining the data record signatures for said learning dataset; and
repeating the selecting a subset of the dataset and determining the data record signatures until a predefined limit is reached.

24. The method of claim 22, further comprising
changing parameters used in determination of data record signatures and performing the determination of data record signatures with the changed parameters; and
repeating the changing of the parameters and performing the determination of data record signatures until a predefined limit is reached.

25. The method of claim 22, further comprising pre-processing the dataset prior to analysing the dataset, said pre-processing comprising
determining the univariate counts for each field in the dataset,
determining the bivariate counts for certain data field pairs of records of the dataset, and
storing the univariate counts and bivariate counts for future use.

26. The method of claim 22, wherein determining the univariate counts comprises
collecting distinct field values for each field in the dataset to obtain a value range for each field; and
calculating for each field a total number of occurrences of each field value in the value range to obtain the univariate counts for each field.

27. The method of claim 22, wherein determining the bivariate counts comprises processing the data fields of the data records in pairs by
collecting distinct field value pairs for each field pair to obtain a bivariate range of the field pair; and
calculating for each field pair a total number of occurrences of each field value in the bivariate value range to obtain bivariate counts for each field pair.

28. The method of claim 22, wherein determining the data record signatures comprises
using a function of the univariate and bivariate counts to determine scores indicative of likelihood of a certain value of the target field and certain values of the explanatory fields to exist in the same data record and using the scores to determine the signatures for different values of the target field.

29. The method of claim 22, wherein said request to provide the predicted field value comprises an indication of the explanatory fields to be used in said prediction.

30. The method of claim 22, wherein said request to provide the predicted field value comprises said predefined limit for repeating said determining of signatures.

31. The method of claim 22, wherein said predefined limit is certain number of iterations or certain score value for the signature.

32. An apparatus comprising
a processor;
a memory including computer program code; the memory and the computer program code configured to, with the processor, cause the apparatus to
analyse a dataset comprising a plurality of data records, wherein each data record comprises data fields, a data field comprising a field name and a field value;
receive a request to provide a predicted field value for a certain target field of a certain target data record, wherein said certain target data record comprises one or more explanatory fields with explanatory values;
determine univariate counts indicative of value variation in said target field and in one or more explanatory fields across the dataset;
determine bivariate counts indicative of value pair variation in field pairs comprising said target field and at least one of the explanatory fields across the dataset;
use the univariate counts and bivariate counts for determining data record signatures for different values of the target field, wherein the signature comprises values of the explanatory fields and wherein determining the data record signatures comprises
determining, for different target field values, a false predictions count indicative of the number of records in the dataset, which records comprise explanatory field names and explanatory field values of a certain signature but which records do not comprise the respective target field value,
determining, for different target field values, a missed predictions count indicative of the number of records in the dataset, which records comprise a certain target field value but do not comprise the respective signature, and
using a function of the false prediction count and missed prediction count for determining the data record signatures;
repeat said determining of signatures until certain predefined limit is reached;
select a signature that at least partially matches values of explanatory fields of the target data record; and
conclude that the predicted field value for the target field is the value of the target field corresponding to the selected signature.

33. The apparatus of claim 32, wherein the memory and the computer program code are further configured to, with the processor, cause the apparatus to
select a subset of the dataset to form a learning dataset comprising a plurality of records and determining the data record signatures for said learning dataset; and
repeat the selecting a subset of the dataset and determining the data record signatures until a predefined limit is reached.

34. The apparatus of claim 32, wherein the memory and the computer program code are further configured to, with the processor, cause the apparatus to
change parameters used in determination of data record signatures and performing the determination of data record signatures with the changed parameters; and
repeat the changing of the parameters and performing the determination of data record signatures until a predefined limit is reached.

35. The apparatus of claim 32, wherein the memory and the computer program code are further configured to, with the processor, cause the apparatus to pre-process the dataset prior to analysing the dataset, wherein the pre-processing comprises
determining the univariate counts for each field in the dataset,
determining the bivariate counts for certain data field pairs of records of the dataset, and
storing the univariate counts and bivariate counts for future use.

36. The apparatus of claim 32, wherein the memory and the computer program code are further configured to, with the processor, cause the apparatus to determine the univariate counts by
collecting distinct field values for each field in the dataset to obtain a value range for each field; and
calculating for each field a total number of occurrences of each field value in the value range to obtain the univariate counts for each field.

37. The apparatus of claim 32, wherein the memory and the computer program code are further configured to, with the processor, cause the apparatus to determine the bivariate counts comprises processing the data fields of the data records in pairs by
collecting distinct field value pairs for each field pair to obtain a bivariate range of the field pair; and
calculating for each field pair a total number of occurrences of each field value in the bivariate value range to obtain bivariate counts for each field pair.

38. The apparatus of claim 32, wherein the memory and the computer program code are further configured to, with the processor, cause the apparatus to determine the data record signatures by
using a function of the univariate and bivariate counts to determine scores indicative of likelihood of a certain value of the target field and certain values of the explanatory fields to exist in the same data record and using the scores to determine the signatures for different values of the target field.

39. A computer program comprising computer executable program code configured to control an apparatus, when the computer executable program code is executed, to
analyse a dataset comprising a plurality of data records, wherein each data record comprises data fields, a data field comprising a field name and a field value;
receive a request to provide a predicted field value for a certain target field of a certain target data record, wherein said certain target data record comprises one or more explanatory fields with explanatory values;
determine univariate counts indicative of value variation in said target field and in one or more explanatory fields across the dataset;
determine bivariate counts indicative of value pair variation in field pairs comprising said target field and at least one of the explanatory fields across the dataset;
use the univariate counts and bivariate counts for determining data record signatures for different values of the target field, wherein the signature comprises values of the explanatory fields and wherein determining the data record signatures comprises
determining, for different target field values, a false predictions count indicative of the number of records in the dataset, which records comprise explanatory field names and explanatory field values of a certain signature but which records do not comprise the respective target field value,
determining, for different target field values, a missed predictions count indicative of the number of records in the dataset, which records comprise a certain target field value but do not comprise the respective signature, and
using a function of the false prediction count and missed prediction count for determining the data record signatures;
repeat said determining of signatures until certain predefined limit is reached;
select a signature that at least partially matches values of explanatory fields of the target data record; and
conclude that the predicted field value for the target field is the value of the target field corresponding to the selected signature.

40. A computer program of claim 39, further comprising computer executable program code configured to control an apparatus, when the computer executable program code is executed, to
select a subset of the dataset to form a learning dataset comprising a plurality of records and determining the data record signatures for said learning dataset; and repeat the selecting a subset of the dataset and determining the data record signatures until a predefined limit is reached.

41. A computer program of claim 39, further comprising computer executable program code configured to control an apparatus, when the computer executable program code is executed, to
determine the data record signatures by using a function of the univariate and bivariate counts to determine scores indicative of likelihood of a certain value of the target field and certain values of the explanatory fields to exist in the same data record and using the scores to determine the signatures for different values of the target field.
